(19) United States
(12) Patent Application Publication
(10) Pub. No.: US 2010/0216897 A1
(43) Pub. Date: Aug. 26, 2010

(54) PROCESS FOR THE PREPARATION OF DIMETHYL ETHER

(76) Inventor: Thomas Rostrup-Nielsen, Holte (DK)

Correspondence Address: DICKSTEIN SHAPIRO LLP, 1825 EYE STREET NW, Washington, DC, 20006-5403, US

(21) Appl. No.: 12/663,058

(22) PCT Filed: Jul. 10, 2008
(86) PCT No.: PCT/EP2008/005625

§ 371 Date: Dec. 4, 2009

(30) Foreign Application Priority Data
Jul. 12, 2007 (DK) PA 2007 01027

(51) Int. Cl. C07C 31/00

(52) U.S. Cl. 518/700

(57) ABSTRACT

Process for the preparation of dimethyl ether by catalytic conversion of synthesis gas to dimethyl ether comprising contacting a stream of synthesis gas comprising carbon dioxide with one or more catalysts active in the formation of methanol and the dehydration of methanol to dimethyl ether, to form a product mixture comprising the components dimethyl ether, carbon dioxide and unconverted synthesis gas, washing the product mixture comprising carbon dioxide and unconverted synthesis gas in a first scrubbing zone with a first solvent rich in dimethyl ether and subsequently washing the effluent from the first scrubbing zone in a second scrubbing zone with a second solvent rich in methanol to form a vapour stream comprising unconverted synthesis gas stream with reduced content of carbon dioxide, transferring the vapour stream comprising unconverted synthesis gas stream with reduced carbon dioxide content for further processing to dimethyl ether.

The invention concerns a process for preparation of dimethyl ether from synthesis gas. In particular, the invention concerns an improved dimethyl ether synthesis process utilising synthesis gas having reduced carbon dioxide content.

### BACKGROUND OF THE INVENTION

The process of the invention concerns the preparation of dimethyl ether in a single-step process from synthesis gas having a reduced content of carbon dioxide.

In the single-step process the conversion of synthesis gas to dimethyl ether is carried out in a single reactor in which synthesis gas is first catalytically converted to methanol, shown in equation (1), followed by dehydration of methanol to dimethyl ether as shown in equation (2). The shift reaction also takes place and is shown in equation (3).

CO+2H₂→CH₃OH (1)

2CH₃OH→CH₃OCH₃+H₂O (2)

CO+H₂O→CO₂+H₂ (3)

The overall reaction for the synthesis gas conversion process to dimethyl ether is given by equation (4).

3H₂+3CO→CH₃OCH₃+CO₂ (4)

Maximum conversion of synthesis gas is obtained when dimethyl ether is prepared at a stoichiometric ratio between hydrogen and carbon monoxide equal to one. At ratios above or below one less dimethyl ether is prepared. However, at a stoichiometric ratio of one, equimolar amounts of dimethyl ether and carbon dioxide are formed according essentially to equation (4).

Carbon dioxide is soluble in dimethyl ether, and in order to obtain the dimethyl ether product it is therefore necessary to remove the carbon dioxide formed. Additionally, when carbon dioxide is removed the composition of the unconverted synthesis gas, which is recycled to the dimethyl ether synthesis reactor, is close to that of the make up synthesis gas used to prepare dimethyl ether, which is an additional advantage. Removal of carbon dioxide from the dimethyl ether product downstream the synthesis reactor can become very costly.

Three basic processes for disposing of carbon dioxide are known. In the first process dimethyl ether is synthesized in a single reactor according to reaction (1) to (3) above. A mixed effluent stream comprising unreacted synthesis gas together with any carbon dioxide present is then separated from the dimethyl ether product, which also contains some unreacted methanol. The separated synthesis gas and carbon dioxide stream is recycled to the synthesis gas process stream entering the reactor. This process is usually used in a hydrogen rich synthesis gas having for instance a ratio between hydrogen and carbon monoxide above 5.

In the second known process a mixed effluent stream comprising unreacted synthesis gas together with any carbon dioxide present is also separated from the dimethyl ether product. However, carbon dioxide is then separated from the synthesis gas. This can be done by washing this stream with for instance a suitable amine compound such as methyl diethanol amine, MDEA. The synthesis gas stream which is free of carbon dioxide is then recycled to the synthesis gas process stream entering the reactor. The carbon dioxide obtained may then be employed in other processes for instance in the preparation of synthesis gas from natural gas by autothermal reforming.

In the third known process only synthesis gas is separated from the dimethyl ether product and carbon dioxide. The dimethyl ether product thus contains both methanol and carbon dioxide. The separated synthesis gas is recycled to the synthesis gas process stream entering the reactor.

Various solvents are known in the prior art for removing carbon dioxide from mixtures with synthesis gas. The choice of solvent is dependent on the ability to dissolve dimethyl ether and carbon dioxide and the ideal solvent should have a high solubility for carbon dioxide and a low volatility.

U.S. Pat. No. 5,908,963 discloses a process for the preparation of dimethyl ether from synthesis gas in which synthesis gas is separated from dimethyl ether product and recycled to the synthesis gas process stream entering the dimethyl ether synthesis loop. The presence of excess methanol in the dimethyl ether product is the focus of the disclosed process and the removal of carbon dioxide is not addressed.

U.S. Pat. No. 6,458,856 discloses a one-step catalytic conversion process for dimethyl ether preparation. After catalytic conversion of synthesis gas to dimethyl ether the effluent from the reactor is separated into a vapour mixture comprising dimethyl ether, carbon dioxide and unconverted synthesis gas. The vapour mixture is scrubbed using a scrubbing solvent to remove dimethyl ether and carbon dioxide from unconverted synthesis gas. The scrubbing solvent comprises a mixture of dimethyl ether and methanol. The unconverted synthesis gas is recycled to the dimethyl reactor.

This reference also discloses prior art in which scrubbing solvents such as methanol, water, methanol/water mixtures, dimethyl ether or ethanol are used.

The English abstract and electronic translation of JP patent application No. 200491327 A disclose a process for the separation of dimethyl ether comprising reacting carbon monoxide with hydrogen to obtain a product gas containing at least dimethyl ether, carbon dioxide and unreacted gas components, cooling the product gas at −10 to −60° C. at 1 to 30 MPa to obtain a liquid phase of dimethyl ether and carbon dioxide and a gas phase containing unreacted gas. It is recommended that if the product gas contains water and methanol these are separated first, preferably by cooling to 0 to 60° C. at 2 to 7 MPa.

The product gas is contacted with liquid dimethyl ether and methanol to absorb dimethyl ether and carbon dioxide contained in the product gas prior to or during condensation of dimethyl ether in the product gas. Dimethyl ether and methanol are effectively supplied such that they may also be contacted with the product gas after condensation, that is, with the gas phase containing the unreacted gas. Either dimethyl ether or methanol may be supplied first or both may be supplied simultaneously. Furthermore, they may be premixed and the mixture may then be supplied.

Dimethyl ether is a good solvent for carbon dioxide but is very volatile, whereas methanol is a poorer solvent for carbon dioxide than dimethyl ether but has the advantage of being less volatile. A process for preparing dimethyl ether from synthesis gas which makes use of a solvent having high solubility for carbon dioxide and simultaneously low volatility is therefore desirable.

It is an objective of the invention to provide a process whereby dimethyl ether production is maximised by recovering dimethyl ether from the obtained waste streams.

It is a further objective of the invention to provide a process whereby methanol is recovered from the dimethyl ether production process and is suitable for further processing.

### BRIEF SUMMARY OF THE INVENTION

The invention relates to a process for the preparation of dimethyl ether by catalytic conversion of synthesis gas to dimethyl ether comprising contacting a stream of synthesis gas comprising carbon dioxide with one or more catalysts active in the formation of methanol and the dehydration of methanol to dimethyl ether, to form a product mixture comprising the components dimethyl ether, carbon dioxide and unconverted synthesis gas, washing the product mixture comprising carbon dioxide and unconverted synthesis gas in a first scrubbing zone with a first solvent rich in dimethyl ether and subsequently washing the effluent from the first scrubbing zone in a second scrubbing zone with a second solvent rich in methanol to form a vapour stream comprising unconverted synthesis gas stream with reduced content of carbon dioxide, transferring the vapour stream comprising unconverted synthesis gas stream with reduced carbon dioxide content for further processing to dimethyl ether.

The process of the invention encompasses recovery of methanol from the used solvents sufficient for further processing in a dimethyl ether reactor. These amounts would ordinarily be regarded as lost.

Furthermore the process of the invention includes maximising production of dimethyl ether by recovering dimethyl ether from the waste streams obtained.

The process of the invention also includes the removal of water from the dimethyl ether product. Water inhibits dimethyl ether production and is also harmful to the catalyst.

### BRIEF DESCRIPTION OF THE DRAWINGS

FIG. 1 shows the general process steps in the preparation of dimethyl ether from synthesis gas.

FIG. 2 shows an embodiment in which the product mixture is washed in accordance with the process of the invention.

FIG. 3 shows an embodiment in which dissolved gases from the washing step are recovered.

FIG. 4 shows an embodiment in which recovery of the used solvent with products without dissolved gasses is carried out.

FIG. 5 shows another embodiment in which dimethyl ether is recovered after washing of the product mixture.

FIG. 6 shows an embodiment of the invention in which water is removed from the process.

FIG. 7 shows an embodiment of the invention in which methanol entering the second scrubbing zone has been treated to remove any dimethyl ether.

FIG. 8 shows another embodiment of the invention.

FIG. 9 shows the effect of using methanol only as a scrubbing solvent.

FIG. 10 shows the effect of using dimethyl ether only as a scrubbing solvent.

FIG. 11 shows the effect of using a mixture of methanol and dimethyl ether as a scrubbing solvent.

FIG. 12 shows the effect of using methanol and dimethyl ether separately only as scrubbing solvents.

FIG. 13 shows a comparison between different solvents.

### DETAILED DESCRIPTION OF THE INVENTION

The invention concerns an improved dimethyl ether synthesis process utilising synthesis gas having reduced carbon dioxide content.

The product mixture from the dimethyl ether synthesis reactor comprises primarily the product dimethyl ether, some unconverted synthesis gas and carbon dioxide. The product mixture from the dimethyl ether synthesis reactor is washed in two consecutive scrubbing zones. Optionally, the product mixture may be separated prior to the wash to provide a gaseous stream comprising unconverted synthesis gas, carbon dioxide and dimethyl ether and a liquid stream comprising carbon dioxide, dimethyl ether and containing minor amounts of methanol and water. Either the thereby obtained vapour stream or the entire product mixture is subjected to the wash.

In the first scrubbing zone the vapour stream or the product mixture is scrubbed with a first solvent rich in dimethyl ether. Dimethyl ether is a suitable solvent for carbon dioxide and carbon dioxide is thus removed from the vapour stream or the product mixture when it is contacted with the dimethyl ether. Since dimethyl ether is very volatile, the vapour stream comprising unconverted synthesis gas or the product stream also contains some evaporated dimethyl ether on leaving the first scrubbing zone.

The evaporated dimethyl ether is removed from the vapour stream or the product mixture by subsequent scrubbing with a second solvent rich in methanol in the second scrubbing zone. Methanol has a high solubility towards dimethyl ether so the evaporated dimethyl ether present in the vapour stream or the product mixture dissolves in the methanol in the second washing zone. Due to the low volatility of methanol the vapour stream or the product mixture leaving the second scrubbing zone does not contain any methanol vapour.

The scrubbed vapour stream or product mixture thereafter comprises unconverted synthesis gas having reduced carbon dioxide content when compared to before scrubbing.

Carrying out the washing process in two separate scrubbing zones with dimethyl ether and methanol, respectively, is advantageous because carbon dioxide is effectively removed from the product mixture or the vapour stream comprising unconverted synthesis gas and any dimethyl ether present in the vapour stream or the product mixture is also effectively removed. The vapour stream or the product mixture comprises therefore only unconverted synthesis gas after scrubbing and it can be recycled to the dimethyl ether synthesis step or be used in other process steps requiring synthesis gas that is not contaminated with carbon dioxide or dimethyl ether.

The scrubbing process is carried out in two separate scrubbing zones which can be placed in either a single scrubbing column or in two separate scrubbing columns in series having at least one layer of conventional trays or packing elements such as raschig rings or others known in the art, in each scrubbing column.

The recycle ratio R/M is defined as the amount, R, of dimethyl ether, methanol or carbon dioxide and unconverted synthesis gas recycled from the second scrubbing zone to the dimethyl ether synthesis reactor, relative to the amount, M, of make-up synthesis gas entering the dimethyl ether synthesis reactor for conversion.

The process of the invention provides a reduction in the amount of dimethyl ether and carbon dioxide in the washed unconverted synthesis gas. A reduction in these compounds leads to a decrease in R and thus to a lower R/M value.

This is advantageous because a reduction in the dimethyl ether and carbon dioxide content in the recycled unconverted synthesis gas, leads to a reduction in the number of recycles and thus to a reduction in the number of passes through the dimethyl ether synthesis reactor. A higher conversion of synthesis gas is therefore obtained.

Preferable is a recycle ratio of DME, methanol, carbon dioxide and unconverted synthesis gas relative to make-up synthesis gas of 1 to 2.

The first solvent rich in dimethyl ether comprises more than 50 mole % and at most 100 mole % dimethyl ether. Preferably, the first solvent comprises at least 80 mole % and at most 100 mole % dimethyl ether. The first solvent can also comprise methanol, dissolved gases etc.

The second solvent rich in methanol comprises more than 50 mole % and at most 100 mole % methanol. Preferably, the second solvent comprises at least 80 mole % and at most 100 mole % methanol. The second solvent can also comprise dimethyl ether, dissolved gases etc.

The ratio of the molar flow of the first solvent rich in dimethyl ether to the flow of the second solvent rich in methanol is in the range of 40:1 to 1:40. Preferably, the range is 20:1 to 1:20. Most preferably, the range is 10:1 to 2:1.

The invention includes the following embodiments:

Process for the preparation of dimethyl ether by catalytic conversion of synthesis gas to dimethyl ether comprising contacting a stream of synthesis gas comprising carbon dioxide with one or more catalysts active in the formation of methanol from synthesis gas and the dehydration of methanol to dimethyl ether, to form a product mixture 3 comprising the components dimethyl ether, carbon dioxide and unconverted synthesis gas, washing the product mixture 3 comprising dimethyl ether, carbon dioxide and unconverted synthesis gas in a first scrubbing zone 4A of washing unit 4 with a first solvent 10 rich in dimethyl ether, and subsequently washing the effluent from the first scrubbing zone 4A in a second scrubbing zone 4B of washing unit 4 with a second solvent 11 rich in methanol to form a vapour stream 5 comprising unconverted synthesis gas with reduced content of carbon dioxide, transferring the vapour stream 5 comprising unconverted synthesis gas with reduced carbon dioxide content for further processing to dimethyl ether.

In an embodiment of the invention, product mixture 3 is separated prior to washing, into a vapour phase 13 comprising unconverted synthesis gas and carbon dioxide, and a liquid phase 14 comprising dimethyl ether, dissolved carbon dioxide, methanol and water, washing vapour phase 13 in first and second scrubbing zones 4A and 4B and withdrawing an additional stream comprising used solvent 12 with dimethyl ether product, methanol and dissolved gases from washing unit 4.

In an embodiment of the invention, the additional stream comprising used solvent 12 is separated in separation unit 16 into solvent 24 comprising dimethyl ether and carbon dioxide and dissolved gases 17 comprising hydrogen and carbon monoxide, sending dissolved gases 17 to first scrubbing zone 4A and optionally withdrawing a side stream 17a from dissolved gases 17 and transferring side stream 17a to second scrubbing zone 4B.

In an embodiment of the invention, either the additional stream comprising used solvent 12 with dimethyl ether product, methanol and dissolved gases, or the solvent 24 containing dimethyl ether and carbon dioxide, is further separated in separation unit 25 to a carbon dioxide comprising stream (26, 26a) and a dimethyl ether rich stream 27, which is further separated in separation unit 29 into dimethyl ether product 31 and impurities 30.

In an embodiment of the invention, a portion (28, 28a) of dimethyl ether rich stream 27 is diverted to first scrubbing zone 4A and/or a portion 32 of dimethyl ether product 31 is sent to first scrubbing zone 4A.

In an embodiment of the invention, the stream with solvent 24 is separated in either one or two consecutive separation units 33,25 into a carbon dioxide comprising stream 26 and a dimethyl ether-rich stream 27, and the dimethyl ether-rich stream 27 is further separated in separation unit 29 into dimethyl ether product 31 and methanol, water and impurities 30.

In an embodiment of the invention, a side stream 37a is removed from separation unit 25 and/or DME portion 37 is removed from dimethyl ether rich stream 27 and/or DME portion 38 is removed from dimethyl ether product 31 and transferred to first scrubbing zone 4A.

In an embodiment of the invention, liquid phase 14 comprising dimethyl ether, dissolved carbon dioxide, methanol and water is separated in separation unit 39 into a water-comprising stream 40 comprising methanol and impurities and a gaseous stream 41, and separating the water-comprising stream 40 in separation unit 42 into a stream of water 43 and a stream of methanol and impurities 44, and catalytically converting the stream of methanol and impurities 44 to dimethyl ether in a dimethyl ether synthesis reactor 2, 2a.

In an embodiment of the invention, a portion of the stream with methanol, water and impurities 30 is diverted to separation unit 42 as stream 30b for separation into a stream of water 43 and a stream of methanol and impurities 44.

In an embodiment of the invention, solvent 12 with dimethyl ether product, methanol and dissolved gases is transferred from first washing zone 4A to separation unit 50 for removal of carbon dioxide comprising stream 26, separating and returning a dimethyl ether rich stream 27 from separation unit 50 to washing zone 4A for further use.

In an embodiment of the invention, a side stream 45 is removed from second scrubbing zone 4B and sent to separator 46 for separation to dimethyl ether 47 and methanol 48, and methanol 48 is transferred to second washing zone 4B and optionally dimethyl ether 47 is combined with carbon dioxide comprising stream 26.

In an embodiment of the invention, the dimethyl ether stream is further separated into a second vapour stream comprising dimethyl ether and a second stream of methanol and the second stream of methanol is thereafter catalytically converted to dimethyl ether.

In an embodiment of the invention, the stream of methanol is mixed with the second stream of methanol prior to catalytic conversion to dimethyl ether.

In an embodiment of the invention, the stream of methanol is used to wash the vapour stream comprising unconverted synthesis gas and carbon monoxide.

In an embodiment of the invention, dimethyl ether obtained by catalytic conversion of the stream of methanol mixed with the second stream of methanol is used to wash the vapour stream comprising unconverted synthesis gas and carbon monoxide.

In an embodiment of the invention, the ratio of the molar flow of the first solvent rich in dimethyl ether to the flow of the second solvent rich in methanol is in the range of 40:1 to 1:40.

In an embodiment of the invention, the ratio of the molar flow of the first solvent rich in dimethyl ether to the flow of the second solvent rich in methanol is in the range of 20:1 to 1:20.

In an embodiment of the invention, the first and second scrubbing zones are either in separate scrubbing columns in series or in a single scrubbing column.

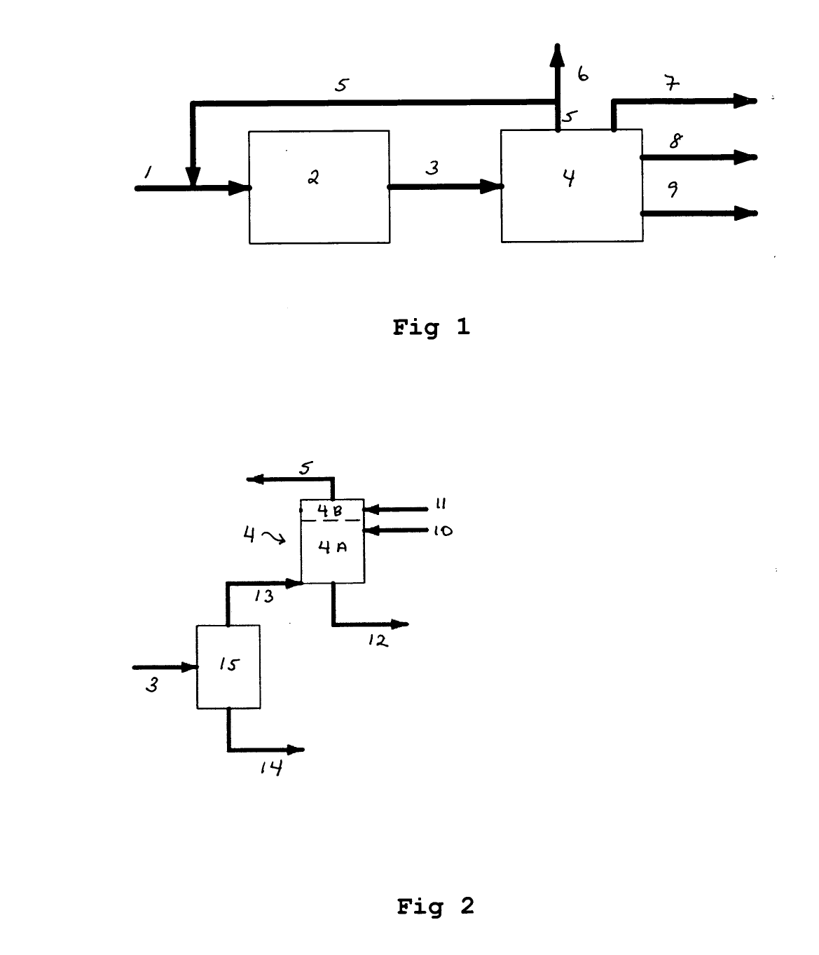

FIG. 1 shows the general process steps in the preparation of dimethyl ether from synthesis gas.

Synthesis gas 1 is sent to DME synthesis reactor 2 for catalytic conversion to methanol and DME according to reactions (1) and (2). The shift reaction also takes place according to reaction (3). The effluent from DME synthesis reactor 2 contains product mixture 3, which comprises a mixture of dimethyl ether, carbon dioxide and unconverted synthesis gas. Product mixture 3 is sent to washing unit 4 for separation of the product dimethyl ether 9 from carbon dioxide 8. A recycle stream 5 comprising unconverted synthesis gas, from which carbon dioxide 8 has been removed, is transferred from washing unit 4 to the stream of synthesis gas 1. Recycle stream 5 may be purged if necessary and is shown as recycle purge stream 6. In addition there may be purge streams 7 from washing unit 4. Inert components such as nitrogen, methane and other components not relevant for the process are removed as inerts.

FIG. 2 shows an embodiment in which the product mixture is washed in accordance with the process of the invention.

Embodiments showing optional separation of the product mixture phase before transfer to the washing unit are included.

The product mixture 3 from DME synthesis reactor 2 comprises a mixture of dimethyl ether, carbon dioxide, unconverted synthesis gas, water and methanol in various amounts. Product mixture 3 is washed in washing unit 4 which comprises first scrubbing zone 4A with a first solvent 10 rich in dimethyl ether to remove carbon dioxide and dimethyl ether and second scrubbing zone 4B for removal of remaining amounts of dimethyl ether. The effluent from the first scrubbing zone 4A comprising unconverted synthesis gas and some evaporated dimethyl ether is washed in second scrubbing zone 4B with a second solvent 11 rich in methanol for dissolution of dimethyl ether in methanol.

This results in a vapour stream 5 comprising unconverted synthesis gas with reduced content of carbon dioxide, which is recycled from second scrubbing zone 4B to the stream of synthesis gas 1 as recycle stream 5. The vapour stream has been cleaned since the carbon dioxide content is reduced. An additional stream comprising used solvent 12 with products and dissolved gases is also removed from washing unit 4. Used solvent 12 comprises methanol and the product dimethyl ether.

Product mixture 3 can optionally first be separated into a vapour phase 13 and a liquid phase 14 in separation unit 15, which can for instance be a flash unit. Vapour phase 13 comprises unconverted synthesis gas and carbon dioxide and is transferred to first scrubbing zone 4A, where it is washed with the first solvent 10 rich in dimethyl ether to remove carbon dioxide followed by washing in the second scrubbing zone 4B with a second solvent 11 rich in methanol for dissolution of dimethyl ether in methanol as described above. This separation step is beneficial because liquid phase 14 comprises dimethyl ether, some dissolved carbon dioxide, methanol and water which can be further separated to obtain valuable amounts of dimethyl ether and methanol.

Both vapour phase 13 and liquid phase 14 can optionally contain some amounts of water.

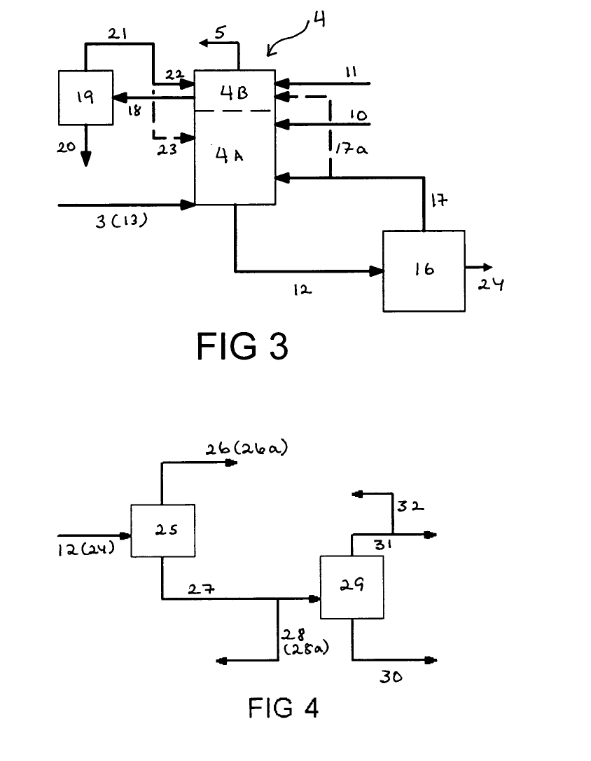

FIG. 3 shows an embodiment in which the product mixture is washed in accordance with the process of the invention and dissolved gases from the washing step are recovered. It is advantageous to recover the dissolved gases since they comprise unconverted synthesis gas. They can further comprise some small amounts of carbon dioxide and inert gases such as nitrogen.

The effluent from DME synthesis reactor 2 consists of product mixture 3, which comprises a mixture of dimethyl ether, carbon dioxide and unconverted synthesis gas. All of product mixture 3 or the portion of the product mixture 3, comprising carbon dioxide and unconverted synthesis gas i.e. vapour phase 13, is transferred to first scrubbing zone 4A and subsequently to second scrubbing zone 4B as described in FIG. 2.

The additional stream comprising used solvent 12 with products and dissolved gases that is removed from washing unit 4 can be transferred to separation unit 16 for removal of dissolved gases 17 comprising carbon monoxide and hydrogen, resulting in solvent 24 containing dimethyl ether and carbon dioxide. It is beneficial to return dissolved gases 17 to first scrubbing zone 4A and/or to second scrubbing zone 4B as stream 17a in order to recover unconverted synthesis gas for recycle to the dimethyl ether synthesis reactor 2.

In addition any remnants of dissolved gases present in second scrubbing zone 4B can also be removed in side stream 18 from second scrubbing zone 4B and thereafter separated in separator 19 into a liquid phase 20 containing solvent with dimethyl ether product and gaseous phase 21 which can be further transferred into second scrubbing zone 4B as stream 22 and/or to the first scrubbing zone 4A as stream 23.

Since the dissolved gases comprise unconverted synthesis gas which is sent to the scrubbing zones for further treatment, the process plant becomes much more effective and the production of dimethyl ether is maximised.

FIG. 4 shows an embodiment in which the product mixture is washed in accordance with the process of the invention and recovery of the used solvent with products and without dissolved gasses is carried out.

The effluent from dimethyl ether synthesis reactor 2 consists of product mixture 3, which comprises a mixture of dimethyl ether, carbon dioxide and unconverted synthesis gas. All of product mixture 3 or the portion of the product mixture 3 comprising carbon dioxide and unconverted synthesis gas, i.e. vapour phase 13, is transferred to first scrubbing zone 4A and subsequently to second scrubbing zone 4B as described in FIG. 2.

The additional stream comprising used solvent 12 with products and dissolved gases that is removed from washing unit 4 can be transferred to separation unit 16 for removal of dissolved gases 17 comprising carbon monoxide and hydrogen resulting in solvent 24 containing DME and carbon dioxide as shown in FIG. 3. This stream can be further treated in order to obtain valuable dimethyl ether, which can be used in washing unit 4 for removal of carbon dioxide or for other purposes.

Dissolved gases 17 can be transferred to first scrubbing zone 4A and optionally a side stream 17a can be withdrawn from dissolved gases 17 and transferred to second scrubbing zone 4B.

In FIG. 4 it can be seen that the stream with solvent 24 containing DME and carbon dioxide is sent to separation unit 25, where carbon dioxide comprising stream 26 is separated from a dimethyl ether-rich stream 27. A portion 28 of dimethyl ether-rich stream 27 can optionally be transferred to scrubbing zones 4A and 4B. Impurities 30 are removed from dimethyl ether-rich stream 27 in separation unit 29 and dimethyl ether product 31 is obtained. This product is suitable for use as a solvent in removal of carbon dioxide, and a portion 32 of the dimethyl ether product 31 can therefore be sent to washing unit 4.

In another embodiment of the invention the additional stream comprising used solvent 12 with products and dissolved gases that is removed from washing unit 4 is not transferred to separation unit 16 for removal of dissolved gases 17 comprising hydrogen and carbon monoxide as shown in FIG. 3. Instead the additional stream comprising used solvent 12 is sent directly to separation unit 25 for separation into a carbon dioxide-comprising stream 26a which also contains dimethyl ether and dissolved gases and into a dimethyl ether rich stream 27. The dimethyl ether rich stream 27 can be transferred directly to first scrubbing zone 4A as stream 28a.

It is therefore possible to remove dissolved gases from the used solvent either in a first separation step in separation unit 16 after the used solvent leaves the scrubbing unit as dissolved gases 17 or in a second separation step in separation unit 25 as carbon dioxide-comprising stream 26a.

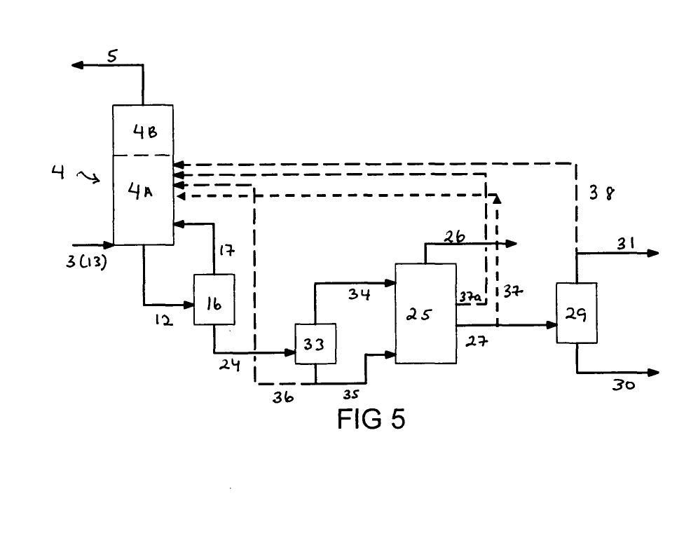

FIG. 5 shows another embodiment in which the product mixture is washed in accordance with the process of the invention and dimethyl ether is thereafter recovered.

The effluent from dimethyl ether synthesis reactor 2 consists of product mixture 3, which comprises a mixture of dimethyl ether, carbon dioxide and unconverted synthesis gas. All of product mixture 3 or the portion of the product mixture 3 comprising carbon dioxide and unconverted synthesis gas i.e. vapour phase 13 is transferred to first scrubbing zone 4A and subsequently to second scrubbing zone 4B as described in FIG. 2.

The stream comprising used solvent 12 with products and dissolved gases that is removed from washing unit 4 can be transferred to separation unit 16 for removal of dissolved gases 17 comprising hydrogen and carbon monoxide resulting in solvent 24 containing DME and carbon dioxide, as described in FIG. 3. This stream can be further treated in order to obtain valuable dimethyl ether, which can be used in first scrubbing zone 4A in washing unit 4 for removal of carbon dioxide as explained in the following:

The stream with solvent 24 contains significant amounts of carbon dioxide. In order to remove this component the stream with solvent 24 can optionally be treated in crude separation unit 33, whereby a gaseous mixture 34 of CO₂ and DME is obtained, and a solvent stream 35 containing dimethyl ether and some small amounts of carbon dioxide is also obtained. A portion 36 of solvent stream 35 can be diverted to first scrubbing zone 4A to provide a source of dimethyl ether. The gaseous mixture 34 of CO₂ and DME and the solvent stream 35 are both sent to separation unit 25 for final separation to a stream of CO₂ 26 and a dimethyl ether-rich stream 27. This stream is sent for further treatment in separation unit 29, whereby methanol, water and impurities 30 are removed and dimethyl ether product 31 is obtained.

A portion can be diverted from either the dimethyl ether-rich stream 27 or the stream of dimethyl ether product 31 or from both these streams as DME portions 37 and 38, respectively, to first scrubbing zone 4A to provide a source of dimethyl ether to washing unit 4. The portion 36 of solvent stream 35 can be diverted to first scrubbing zone 4A to provide a source of dimethyl ether.

In an embodiment of the invention, dimethyl ether rich stream 27 is removed from the bottom of separation unit 25 and sent to separation unit 29 for removal of impurities 30 as described in FIG. 4. Simultaneously, a side stream 37a can be removed from separation unit 25 and sent to scrubbing zone 4A as side stream 37a is rich in dimethyl ether. The removal of side stream 37a allows for the adjustment of the dimethyl ether concentration in dimethyl ether rich stream 27.

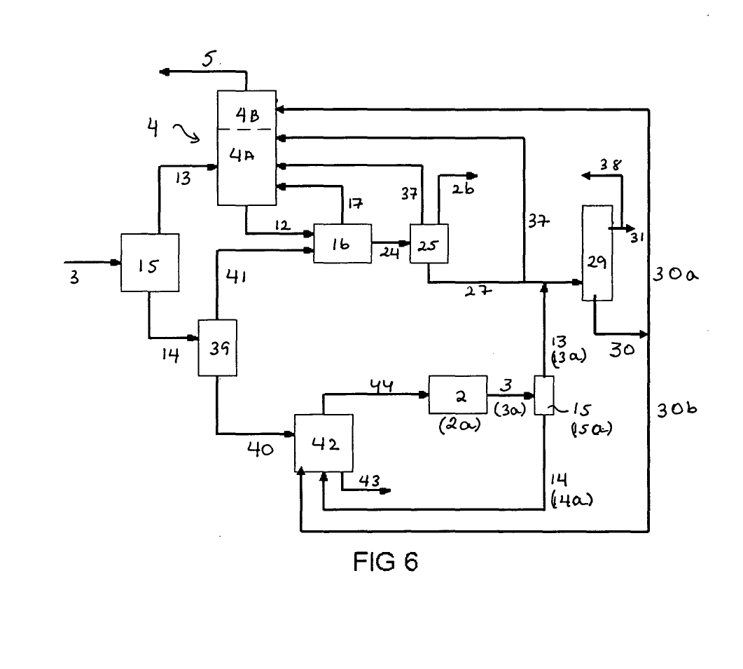

FIG. 6 shows an embodiment of the invention in which water is removed from the process. Water is produced during the dehydration reaction (reaction (4)) of methanol to dimethyl ether, and it is therefore an undesirable component since it can inhibit the reaction. It inhibits the catalyst when present in too large amounts, and it is therefore necessary to remove it.

This embodiment of the invention therefore provides a process whereby water is removed from product mixture 3 and the removed water is clean enough with low methanol content to satisfy environmental requirements.

The effluent from dimethyl ether synthesis reactor 2 consists of product mixture 3, which comprises a mixture of dimethyl ether, carbon dioxide and unconverted synthesis gas. Product mixture 3 is first separated into a vapour phase 13 and a liquid phase 14 in separation unit 15. Liquid phase 14 comprises water.

Vapour phase 13 comprises unconverted synthesis gas and carbon dioxide and is transferred to first scrubbing zone 4A, where it is washed with the first solvent rich in dimethyl ether to remove carbon dioxide followed by washing in the second scrubbing zone 4B with a second solvent 11 rich in methanol for dissolution of dimethyl ether in methanol as described earlier in FIG. 2.

In this embodiment the stream comprising used solvent 12 with products and dissolved gases that is removed from washing unit 4 can be transferred to separation unit 16 for removal of dissolved gases 17 comprising hydrogen and carbon monoxide resulting in solvent 24 containing DME and carbon dioxide. The solvent 24 containing DME and carbon dioxide can be further treated as explained in FIG. 5. Methanol, water and impurities 30 are separated from dimethyl ether-rich stream 27 in separation unit 29, and this stream 30 can either be sent to second scrubbing zone 4B as stream 30a, and optionally a portion can be diverted to separation unit 42 as stream 30b for separation into a stream of water 43 and a stream of methanol and impurities 44.

A portion 37 of dimethyl ether-rich stream 27 can be sent to scrubbing zone 4A.

In the following, the removal of water from liquid phase 14 is described.

Liquid phase 14 is transferred to separation unit 39 for separation into a water-comprising stream 40 that also contains methanol and impurities and a gaseous stream 41 comprising carbon dioxide, dimethyl ether and dissolved gases. The gaseous stream 41 can be sent to separation unit 16 for removal of dissolved gases 17 as described in FIGS. 3 and 5.

The water-comprising stream 40 can be transferred to another separation unit 42 from which two streams are removed: a stream of water 43 and a stream of methanol and impurities 44. The stream of methanol and impurities 44 can be transferred to the dimethyl synthesis reactor 2 for catalytic preparation of more dimethyl ether via dehydration of methanol.

In an embodiment of the invention, the stream of methanol and impurities 44 is transferred to the dimethyl synthesis reactor 2 for preparation of more dimethyl ether via dehydration of methanol. The resulting product mixture comprising dimethyl ether is separated into a vapour phase 13 and a liquid phase 14 in separation unit 15 as described in FIG. 2. Vapour phase 13 comprises unconverted synthesis gas and carbon dioxide and is combined with dimethyl ether-rich stream 27. The combined streams 13 and 27 are sent for further treatment in separation unit 29, whereby methanol, water and impurities 30 are removed and dimethyl ether product 31 is obtained. Liquid phase 14 can be transferred to separation unit 39 as described earlier or to separation unit 42 for separation of a stream of water 43.

Alternatively, the stream of methanol and impurities 44 can be transferred to dimethyl ether synthesis reactor 2a that is locally integrated in the plant and is adapted to convert smaller amounts of methanol to dimethyl ether. Dimethyl ether synthesis reactor 2a can therefore be smaller in size than dimethyl synthesis reactor 2 and can be integrated together with separation unit 15a. Stream 3a comprising dimethyl ether is transferred from dimethyl ether synthesis reactor 2a to separation unit 15a for separation into vapour stream 13a comprising dimethyl ether and liquid phase 14a comprising water. Vapour stream 13a comprising dimethyl ether is combined with dimethyl ether-rich stream 27 for recovery of dimethyl ether product in separation unit 29, while liquid phase 14a is transferred to separation unit 42 for removal of a stream of water 43.

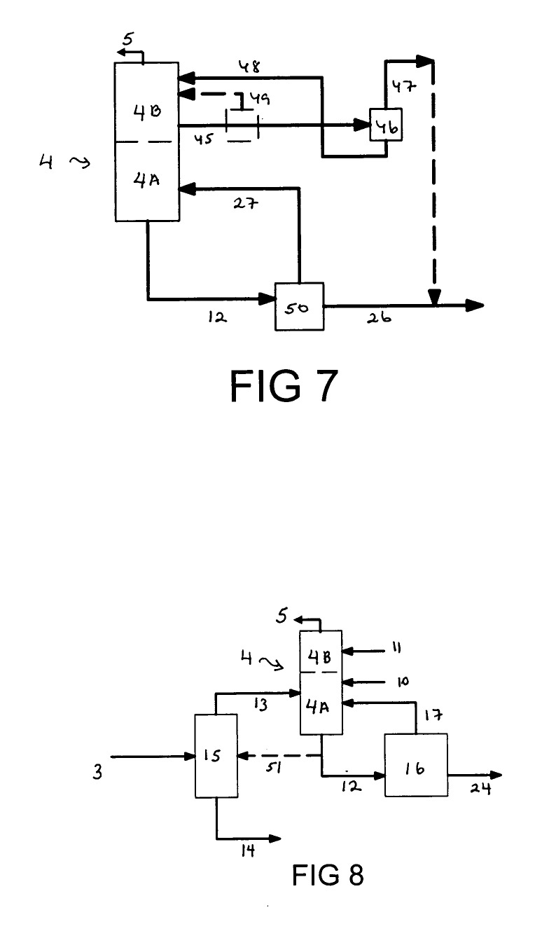

FIG. 7 shows an embodiment of the invention in which the second solvent 11 rich in methanol entering the second scrubbing zone 4B has been treated to remove any dimethyl ether it may contain prior to entry into second scrubbing zone 4B. Volatile dimethyl ether present in scrubbing zone 4B is removed from this zone and combined with carbon dioxide comprising stream 26 for further treatment to obtain dimethyl ether product.

If the washing process carried out in washing unit 4 consists of a single separator, then there may be some dimethyl ether dissolved in the second solvent 11 rich in methanol due to the volatility of the dimethyl ether in the first solvent 10 rich in dimethyl ether.

Removal of dimethyl ether from second solvent 11 rich in methanol prior to entry into second scrubbing zone 4B can be carried out by removing a side stream 45 from second scrubbing zone 4B and sending it to separator 46 for separation to dimethyl ether 47 and methanol 48. Methanol 48 can be returned to second scrubbing zone 4B. A portion 49 can optionally be separated from side stream 45 and returned to second scrubbing zone 4B if required.

Used solvent 12 with products and dissolved gases is transferred from first scrubbing zone 4A to separation unit 50 for removal of carbon dioxide comprising stream 26. A dimethyl ether rich stream 27 can be withdrawn from separation unit 50 and returned to first scrubbing zone 4A for further use.

Separation unit 50 can optionally comprise separation unit 16 and/or separation unit 25 as mentioned earlier in the description of FIGS. 4 and 5.

Carbon dioxide comprising stream 26 comprises some dimethyl ether, methanol and water, and this stream can optionally be combined with dimethyl ether 47 for further treatment to obtain dimethyl ether product.

FIG. 8 shows another embodiment of the invention in which water is removed from the used solvent emerging from the scrubbing unit 4.

The effluent from DME synthesis reactor 2 consists of product mixture 3, which comprises a mixture of dimethyl ether, carbon monoxide, unconverted synthesis gas, water and methanol in various amounts. Product mixture 3 is first separated into vapour phase 13 and liquid phase 14 in separation unit 15. Vapour phase 13 comprises unconverted synthesis gas and carbon dioxide and is transferred to first scrubbing zone 4A, where it is washed with the first solvent 10 rich in dimethyl ether to remove carbon dioxide followed by washing in the second scrubbing zone 4B with a second solvent 11 rich in methanol for dissolution of dimethyl ether in methanol as described earlier. This separation step is beneficial, because liquid phase 14 comprises dimethyl ether, some dissolved carbon dioxide, methanol and water which can be further separated to obtain valuable amounts of dimethyl ether and methanol as described in FIG. 6.

The additional stream comprising used solvent 12 with products and dissolved gases that is removed from washing unit 4 is transferred to separation unit 16 for removal of dissolved gases 17 comprising carbon monoxide and hydrogen resulting in used solvent 24 containing DME and carbon dioxide. It is beneficial to return dissolved gases 17 to first scrubbing zone 4A in order to recover unconverted synthesis gas for recycle to the DME synthesis reactor, as explained in FIG. 3. Solvent 24 comprising dimethyl ether and carbon dioxide can be subjected to further processing steps.

Optionally, a side stream 51 can be diverted from used solvent 12 containing products only to the top section of separation unit 15, which contains vapour phase 13. This is advantageous because any water present in vapour phase 13 will be transferred to side stream 51. Water will be removed from vapour phase 13 and will therefore be eliminated in the gas entering first scrubbing zone 4A.

### EXAMPLES

The effect of using different solvents for scrubbing the synthesis gas for removal of carbon dioxide is shown using the process of the invention described in FIG. 8.

### Comparative Example 1

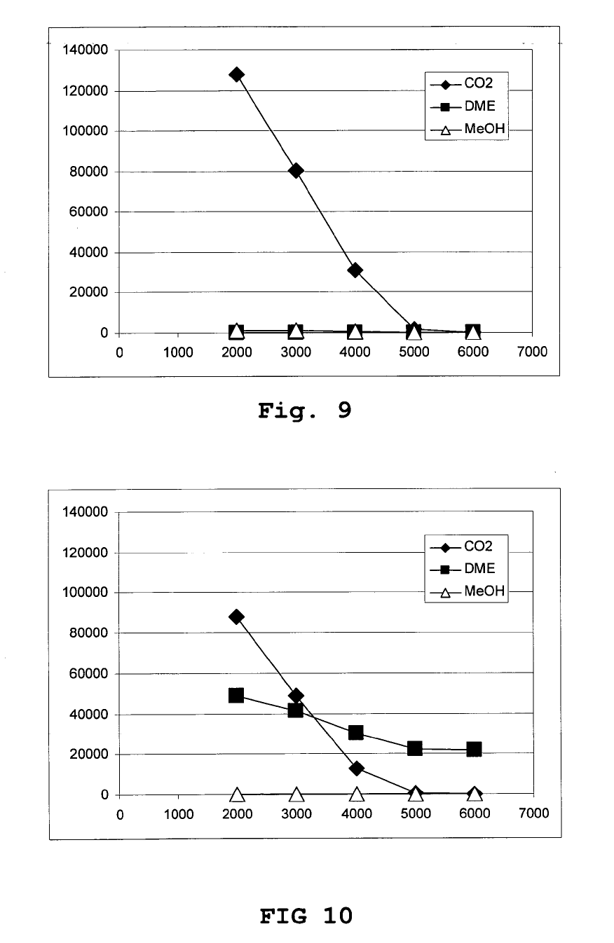

In this example the effect of using methanol only (100% methanol) as a scrubbing solvent is shown. The flow of methanol into the washing unit is given and the content of carbon dioxide (CO₂), dimethyl ether (DME) and methanol (MeOH) in the unconverted synthesis gas recycled from the second scrubbing zone, i.e. in the recycle stream, to the DME synthesis reactor is given in both ppm and percentage in Table 1 and shown in FIG. 9.

In FIG. 8 the flow of methanol into the washing unit 4 is given by stream 11 and the unconverted synthesis gas recycled from second scrubbing zone 4B to DME synthesis reactor 2 is given by stream 5.

TABLE 1

| MeOH | | | | | | |
|---|---|---|---|---|---|---|
| flow | CO₂ | DME | MeOH | CO₂ | DME | MeOH |
| kmol/h | ppm | ppm | ppm | % | % | % |
| 2000 | 127741 | 1 | 1175 | 12.8% | 0.0% | 0.1% |
| 3000 | 80487 | 0 | 846 | 8.0% | 0.0% | 0.1% |
| 4000 | 30989 | 0 | 468 | 3.1% | 0.0% | 0.0% |
| 5000 | 1509 | 0 | 198 | 0.2% | 0.0% | 0.0% |
| 6000 | 63 | 0 | 172 | 0.0% | 0.0% | 0.0% |

From the results it can be seen that using methanol alone as a scrubbing solvent leads to a reduction in the carbon dioxide content in the unconverted synthesis gas. However, it is expected that the reduction in carbon dioxide content could be greater than given if dimethyl ether is used, as it is a more effective solvent of carbon dioxide.

### Comparative Example 2

In this example the effect of using dimethyl ether only (100% DME) as a scrubbing solvent is shown. The flow of DME into the washing unit is given and the content of carbon dioxide (CO₂), dimethyl ether (DME) and methanol (MeOH) in the unconverted synthesis gas recycled from the second scrubbing zone, i.e. in the recycle stream, to the DME synthesis reactor is given in both ppm and percentage in Table 2 and shown in FIG. 10.

In FIG. 8 the flow of dimethyl ether into the washing unit 4 is given by stream 10 and the unconverted synthesis gas recycled from second scrubbing zone 4B to DME synthesis reactor 2 is given by stream 5.

TABLE 2

| DME | | | | | | |
|---|---|---|---|---|---|---|
| flow | CO₂ | DME | MeOH | CO₂ | DME | MeOH |
| kmol/h | ppm | ppm | ppm | % | % | % |
| 2000 | 88248 | 48727 | 0 | 8.8% | 4.9% | 0.0% |
| 3000 | 48655 | 41582 | 0 | 4.9% | 4.2% | 0.0% |
| 4000 | 12649 | 30026 | 0 | 1.3% | 3.0% | 0.0% |
| 5000 | 399 | 22218 | 0 | 0.0% | 2.2% | 0.0% |
| 6000 | 34 | 21521 | 0 | 0.0% | 2.2% | 0.0% |

From the results it can be seen that using dimethyl ether alone as a scrubbing solvent leads to a greater reduction in the carbon dioxide content in the unconverted synthesis gas than if methanol alone is used. A comparison with the results in Table 1 shows that dimethyl ether is more effective than methanol as a carbon dioxide solvent. However there is an amount of dimethyl ether present in the unconverted synthesis gas. This is due to the high volatility of dimethyl ether.

### Comparative Example 3

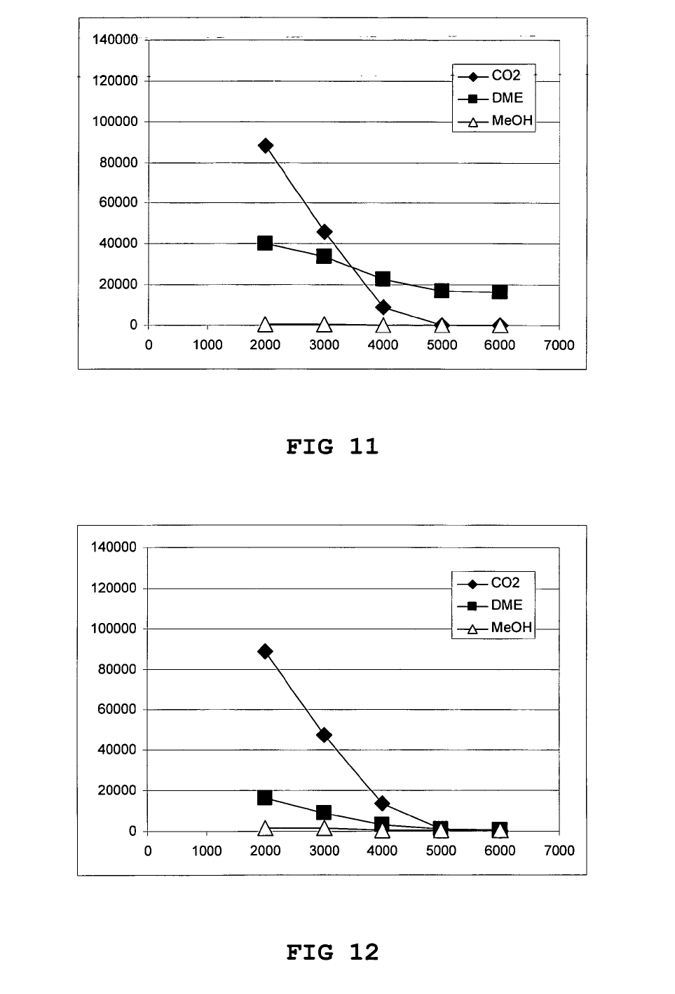

In this example the effect of using a mixture of dimethyl ether and methanol in the ratio 70 DME/30 MeOH as a scrubbing solvent is shown. The flow of the mixture into the washing unit is given and the content of carbon dioxide (CO₂), dimethyl ether (DME) and methanol (MeOH) in the unconverted synthesis gas recycled from the second scrubbing zone, i.e. in the recycle stream, to the DME synthesis reactor is given in both ppm and percentage in Table 3 and shown in FIG. 11.

In FIG. 8 the flow of mixture of dimethyl ether and methanol in the ratio 70 DME/30 MeOH as a scrubbing solvent into the washing unit is given by either stream 10 or 11 and the unconverted synthesis gas recycled from second scrubbing zone 4B to DME synthesis reactor 2 is given by stream 5.

TABLE 3

| Flow of | | | | | | |
|---|---|---|---|---|---|---|
| mixture | CO₂ | DME | MeOH | CO₂ | DME | MeOH |
| kmol/h | ppm | ppm | ppm | % | % | % |
| 2000 | 88049 | 40375 | 398 | 8.8% | 4.0% | 0.0% |
| 3000 | 45720 | 33636 | 281 | 4.6% | 3.4% | 0.0% |
| 4000 | 8746 | 22713 | 136 | 0.9% | 2.3% | 0.0% |
| 5000 | 151 | 16735 | 79 | 0.0% | 1.7% | 0.0% |
| 6000 | 13 | 16338 | 75 | 0.0% | 1.6% | 0.0% |

From the results it can be seen that using a 70 DME/30 MeOH mixture as a scrubbing solvent in the washing unit gives results similar to those obtained when using dimethyl ether only. The carbon dioxide content is similar to that obtained using dimethyl ether only, and the dimethyl ether content in the unconverted synthesis gas stream is only slightly lower than the content obtained using pure dimethyl ether.

### Example 4

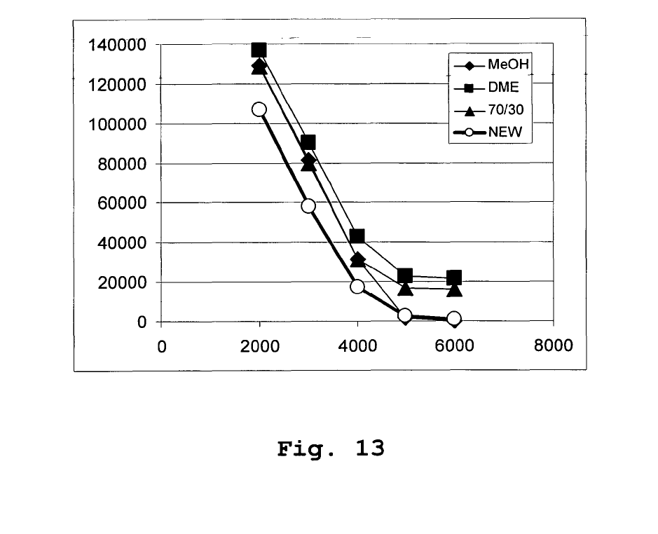

In this example the effect of using both dimethyl ether and methanol as separate scrubbing solvents according to the invention is shown. The flow of the solvents into the washing unit is given and the content of carbon dioxide (CO₂), dimethyl ether (DME) and methanol (MeOH) in the unconverted synthesis gas recycled from the second scrubbing zone, i.e. in the recycle stream, to the DME synthesis reactor is given in both ppm and percentage in Table 4 and shown in FIG. 12.

In FIG. 8 the flow of dimethyl ether as a scrubbing solvent into the washing unit is given by stream 10 and the flow of methanol as a scrubbing solvent into the washing unit is given by stream 11. The unconverted synthesis gas recycled from second scrubbing zone 4B to DME synthesis reactor 2 is given by stream 5.

TABLE 4

| Total | | | | | | | | |
|---|---|---|---|---|---|---|---|---|
| flow | MeOH | DME | CO₂ | DME | MeOH | CO₂ | DME | MeOH |
| kmol/h | kmol/h | kmol/h | ppm | ppm | Ppm | % | % | % |
| 2000 | 400 | 1600 | 88853 | 16184 | 1793 | 8.9% | 1.6% | 0.2% |
| 3000 | 600 | 2400 | 47796 | 8996 | 1331 | 4.8% | 0.9% | 0.1% |
| 4000 | 800 | 3200 | 13492 | 3226 | 670 | 1.3% | 0.3% | 0.1% |
| 5000 | 1000 | 4000 | 1270 | 983 | 338 | 0.1% | 0.1% | 0.0% |
| 6000 | 1200 | 4800 | 171 | 549 | 273 | 0.0% | 0.1% | 0.0% |

Table 5 shows a comparison of the solvents used in Examples 1 to 4. The sum of carbon dioxide, dimethyl ether and methanol in the unconverted synthesis gas recycled from the second scrubbing zone, i.e. in the recycle stream, to the DME synthesis reactor is shown as a function of the solvent flow is shown in FIG. 13.

The results obtained in Table 4, when compared to the results in Table 3 show that using both dimethyl ether and methanol as separate scrubbing solvents according to the invention, leads to a decrease in carbon dioxide content and to a greater reduction in dimethyl ether than obtained using a 70/30 mixture of dimethyl ether and methanol.

TABLE 5

| | 100% MeOH | 100% DME | 70/30 mix | Invention |
|---|---|---|---|---|
| Total flow | Total | Total | Total | Total |
| kmol/h | ppm | ppm | ppm | ppm |
| 2000 | 128917 | 136975 | 128822 | 106830 |
| 3000 | 81333 | 90237 | 79637 | 58123 |
| 4000 | 31457 | 42675 | 31595 | 17388 |
| 5000 | 1707 | 22617 | 16965 | 2591 |
| 6000 | 235 | 21555 | 16426 | 993 |

The results of Table 5 show that the total amount of carbon dioxide, dimethyl ether and methanol is lowest when using the process of the invention.

1. Process for the preparation of dimethyl ether by catalytic conversion of synthesis gas to dimethyl ether comprising contacting a stream of synthesis gas comprising carbon dioxide with one or more catalysts active in the formation of methanol from synthesis gas and the dehydration of methanol to dimethyl ether, to form a product mixture 3 comprising the components dimethyl ether, carbon dioxide and unconverted synthesis gas, washing the product mixture 3 comprising dimethyl ether, carbon dioxide and unconverted synthesis gas in a first scrubbing zone 4A of washing unit 4 with a first solvent 10 rich in dimethyl ether, and subsequently washing the effluent from the first scrubbing zone 4A in a second scrubbing zone 4B of washing unit 4 with a second solvent 11 rich in methanol to form a vapour stream 5 comprising unconverted synthesis gas with reduced content of carbon dioxide, transferring the vapour stream 5 comprising unconverted synthesis gas with reduced carbon dioxide content for further processing to dimethyl ether.

2. Process according to claim 1, wherein product mixture 3 is separated prior to washing into a vapour phase 13 comprising unconverted synthesis gas and carbon dioxide, and a liquid phase 14 comprising dimethyl ether, dissolved carbon dioxide, methanol and water, washing vapour phase 13 in first and second scrubbing zones 4A and 4B and withdrawing an additional stream comprising used solvent 12 with dimethyl ether product, methanol and dissolved gases from washing unit 4.

3. Process according to claim 2, wherein the additional stream comprising used solvent 12 is separated in separation unit 16 into solvent 24 comprising dimethyl ether and carbon dioxide and dissolved gases 17 comprising hydrogen and carbon monoxide, sending dissolved gases 17 to first scrubbing zone 4A and optionally withdrawing a side stream 17a from dissolved gases 17 and transferring side stream 17a to second scrubbing zone 4B.

4. Process according to claim 3, wherein the stream with solvent 24 is separated in either one or two consecutive separation units 33,25 into a carbon dioxide comprising stream 26 and a dimethyl ether-rich stream 27, and the dimethyl ether-rich stream 27 is further separated in separation unit 29 into dimethyl ether product 31 and methanol, water and impurities 30.

5. Process according to claim 2, wherein liquid phase 14 comprising dimethyl ether, dissolved carbon dioxide, methanol and water is separated in separation unit 39 into a water-comprising stream 40 comprising methanol and impurities and a gaseous stream 41, and separating the water-comprising stream 40 in separation unit 42 into a stream of water 43 and a stream of methanol and impurities 44, and catalytically converting the stream of methanol and impurities 44 to dimethyl ether in a dimethyl ether synthesis reactor 2, 2a.

6. Process according to claim 4, wherein a portion of the stream with methanol, water and impurities 30 is diverted to separation unit 42 as stream 30b for separation into a stream of water 43 and a stream of methanol and impurities 44.

7. Process according to claim 1, wherein the dimethyl ether stream is further separated into a second vapour stream comprising dimethyl ether and a second stream of methanol and the second stream of methanol is thereafter catalytically converted to dimethyl ether.

8. Process according to claim 7, wherein the stream of methanol is mixed with the second stream of methanol prior to catalytic conversion to dimethyl ether.

9. Process according to claim 1, wherein the stream of methanol is used to wash the vapour stream comprising unconverted synthesis gas and carbon monoxide.

10. Process according to claim 8, wherein dimethyl ether obtained by catalytic conversion of the stream of methanol mixed with the second stream of methanol is used to wash the vapour stream comprising unconverted synthesis gas and carbon monoxide.

11. Process according to claim 1, wherein the ratio of the molar flow of the first solvent rich in dimethyl ether to the flow of the second solvent rich in methanol is in the range of 40:1 to 1:40.

12. Process according to claim 11, wherein the ratio of the molar flow of the first solvent rich in dimethyl ether to the flow of the second solvent rich in methanol is in the range of 20:1 to 1:20.

13. Process according to claim 1, wherein the first and second scrubbing zones are either in separate scrubbing columns in series or in a single scrubbing column.
